(19) United States
(12) Patent Application Publication
(10) Pub. No.: US 2024/0318122 A1
(43) Pub. Date: Sep. 26, 2024

(54) CELL SEEDING METHOD AND CELL CULTURE SYSTEM

(71) Applicant: SHIMADZU CORPORATION, Kyoto-shi (JP)

(72) Inventor: Yoichi FUJIYAMA, Kyoto-shi (JP)

(21) Appl. No.: 18/602,884

(22) Filed: Mar. 12, 2024

(30) Foreign Application Priority Data
Mar. 22, 2023 (JP) 2023-045481

(51) Int. Cl. C12M 1/34; C12M 1/00

(52) U.S. Cl. CPC C12M 41/46; C12M 27/18

(57) ABSTRACT

A cell seeding method according to this invention includes a step of filling a flow channel of a cell culture device with a culture medium, the cell culture device including a first port that is selectively fluidly connected to a culture medium supplier through a pipe, a second port, and the flow channel that is provided between the first and second ports; and a step of introducing a cell suspension into the flow channel from the second port side of the flow channel with the first port being connected to the pipe after the step of filling the flow channel with the culture medium.

### CROSS-REFERENCE TO RELATED APPLICATIONS

The related application number JP2023-045481, cell seeding method and cell culture system, 22 Mar. 2023, Yoichi Fujiyama upon which this patent application is based are hereby incorporated by reference

### BACKGROUND OF THE INVENTION

### Field of the Invention

The present invention relates to a cell seeding method and a cell culture system.

### Description of the Background Art

Methods for seeding and cultivating cells in a microfluidic device are known in the art (for example, Patent Document 1). Such a method is disclosed, for example, in International Publication No. WO 2016-172454.

When a pipe that feeds a culture medium is connected to an inlet of a flow channel of a fluid device in cell culture, if the pipe includes air bubbles, the air bubbles will be introduced into the flow channel. In particular, in a case in which a cell suspension is introduced from the inlet of the flow channel, after the pipe connected to the inlet of the flow channel is removed, the cell suspension is introduced from the inlet of the flow channel and the removed pipe is reconnected to the inlet of the flow channel. In this case, air bubbles will be included in the pipe by detachment/attachment of the pipe to/from the inlet of the flow channel, and as a result the air bubbles are likely to be introduced into the flow channel. If the air bubbles introduced are located in proximity to cells, the culture medium cannot be properly supplied to the cells by influences of the air bubbles, and can cause improper cell culture.

### SUMMARY OF THE INVENTION

The present invention is intended to solve the above problem, and one object of the present invention is to provide a cell seeding method and a cell culture system capable of suppressing influences of air bubbles and properly performing cell culture.

A cell seeding method according to a first aspect of the present invention includes a step of filling a flow channel of a cell culture device with a culture medium, the cell culture device including a first port that is selectively fluidly connected to a culture medium supplier through a pipe, a second port, and the flow channel that is provided between the first and second ports; and a step of introducing a cell suspension into the flow channel from the second port side of the flow channel with the first port being connected to the pipe after the step of filling the flow channel with the culture medium.

A cell culture system according to a second aspect of the present invention includes a cell culture device including a first port, a second port, and a flow channel that is provided between the first and second ports; a pipe that is connected to the first port; a culture medium supplier that is selectively fluidly connected to the first port via the pipe; a cell supplier that is selectively fluidly connected to the second port, and a controller, wherein the controller is configured to execute a step of filling the flow channel with a culture medium, and a step of introducing a cell suspension into the flow channel from the second port side of the flow channel with the first port being connected to the pipe after the step of filling the flow channel with the culture medium.

In the cell seeding method according to the first aspect and the cell culture system according to the second aspect, as discussed above, the step of filling the flow channel with the culture medium is provided. Accordingly, air bubbles can be removed by feeding the culture medium when filling the flow channel with the culture medium. In addition, the step of introducing the cell suspension into the flow channel from the second port side of the flow channel with the first port being connected to the pipe after the step of filling the flow channel with the culture medium is provided. As a result, the cell suspension (cells) can be introduced without detachment/attachment of the pipe on the first port side to/from the inlet side of the flow channel dissimilar to in a case in which the cell suspension is introduced from the first port side of the flow channel. Consequently, it is possible to remove air bubbles caused by detachment/attachment of the pipe on the first port side to/from the inlet side of the flow channel. Therefore, it is possible to suppress influences of the air bubbles and properly perform cell culture.

### BRIEF DESCRIPTION OF THE DRAWINGS

FIG. 1 is a schematic diagram entirely showing a configuration of a cell culture system according to a first embodiment.

FIG. 2 is a flowchart illustrating a cell seeding image processing method according to the first embodiment.

FIG. 3 is a schematic diagram illustrating a step of filling a flow channel with a culture medium in the first embodiment.

FIG. 4 is a schematic diagram (1) illustrating a step of introducing a cell suspension into the flow channel in the first embodiment.

FIG. 5 is a schematic diagram (2) illustrating a step of introducing a cell suspension into the flow channel in the first embodiment.

FIG. 6 is a schematic diagram (3) illustrating a step of introducing a cell suspension into the flow channel in the first embodiment.

FIG. 7 is a schematic diagram illustrating cell culture using a cell culture device in the first embodiment.

FIG. 8 is a flowchart illustrating a cell seeding image processing method according to a modified example of the first embodiment.

FIG. 9 is a schematic diagram illustrating a step of filling a flow channel with a culture medium in the cell seeding image processing method according to the modified example of the first embodiment.

FIG. 10 is a schematic diagram illustrating a step of attaching a pump to a pipe in the cell seeding image processing method according to the modified example of the first embodiment.

FIG. 11 is a schematic diagram entirely showing a configuration of a cell culture system according to a second embodiment.

### DESCRIPTION OF THE PREFERRED EMBODIMENT

Embodiments embodying the present invention will be described with reference to the drawings.

### First Embodiment

### (Overall Configuration of Cell Culture System)

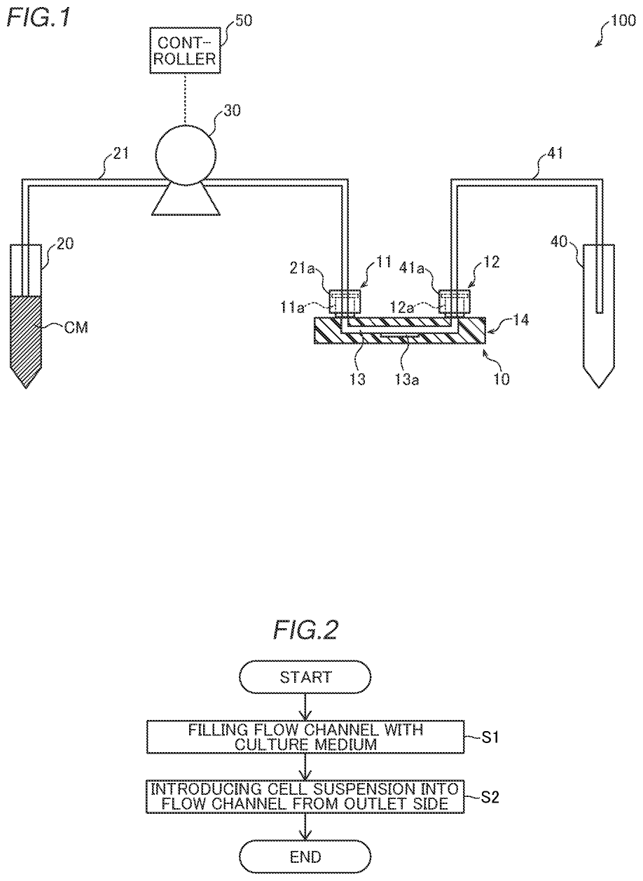

The following description describes a configuration of a cell culture system 100 according to a first embodiment with reference to FIG. 1.

As shown in FIG. 1, the cell culture system 100 is a system for cultivating cells in a cell culture area 13a of a cell culture device 10. The cell culture system 100 includes the cell culture device 10, an inlet-side reservoir 20, a pump 30, an outlet-side reservoir 40, and a controller 50. The inlet-side reservoir 20 and the pump 30 are examples of a “culture medium supplier” in the claims. The outlet-side reservoir 40 is an example of a “second-port side reservoir” in the claims.

The cell culture device 10 includes an inlet 11, an outlet 12 and a flow channel 13. The flow channel 13 is formed between the inlet 11 and the outlet 12, and has a cell culture area 13a. The cell culture area 13a can be the entire flow channel 13, or be a cell culture element for cultivating the cells, which is arranged in the entire flow channel and will be described later. The cell culture element is, for example, a gel (such as collagen gel) or a porous membrane. The cell culture element is arranged in a position in the flow channel 13 that is not directly exposed to the outside. The inlet 11 and the outlet 12 are an example of a “first port” and an example of a “second port” in the claims, respectively.

The inlet 11, the outlet 12, and the flow channel 13 are formed in a device body 14. The device body 14 is formed of a plurality of sheet materials laminated on each other. The plurality of sheet materials have openings or grooves that correspond to the inlet 11, the outlet 12 and the flow channel 13. The inlet 11, the outlet 12 and the flow channel 13 are formed by laminating the plurality of sheet materials on each other. Each of the plurality of sheet materials is formed from a material such as resin, glass or rubber.

The inlet-side reservoir 20 is configured for storage of a culture medium CM supplied to the cell culture device 10. The inlet-side reservoir 20 is a container such as a conical tube. The inlet-side reservoir 20 is selectively fluidly connected to the inlet 11 of the cell culture device 10 through a pipe 21. The pipe 21 is a tube formed from a flexible material such as resin or rubber. One end of the pipe 21 is arranged in the inlet-side reservoir 20, and is immersed in the culture medium CM that is stored in the inlet-side reservoir 20. Another end of the pipe 21 is connected to the inlet 11 of the cell culture device 10. Specifically, a connector 21a is provided on the another end of the pipe 21, and the connector 21a is connected to a connector 11a that is provided on the inlet 11 of the cell culture device 10. For example, the connector 21a is a male luer connector, while the connector 11a is a female luer connector.

The pump 30 transfers the culture medium CM. The pump 30 is provided to the inlet 11 side of the flow channel 13 of the cell culture device 10. Specifically, the pump 30 is attached to the pipe 21. The pump 30 is a tube pump (peristaltic pump) configured to transfer the culture medium CM by pressing the pipe 21. The inlet-side reservoir 20 and the pump 30 serve as a culture medium supplier for supplying the culture medium CM to the cell culture device 10.

The outlet-side reservoir 40 is configured for storage of the culture medium CM that is discharged from the cell culture device 10. The outlet-side reservoir 40 is a container such as a conical tube. The outlet-side reservoir 40 is selectively fluidly connected to the outlet 12 of the cell culture device 10 through a pipe 41. The pipe 41 is a tube formed from a flexible material such as resin or rubber. One end of the pipe 41 is arranged in the outlet-side reservoir 40. Another end of the pipe 41 is connected to the outlet 12 of the cell culture device 10. Specifically, a connector 41a is provided on the another end of the pipe 41, and the connector 41a is connected to a connector 12a that is provided on the outlet 12 of the cell culture device 10. For example, the connector 41a is a male luer connector, while the connector 12a is a female luer connector.

The controller 50 includes a processor such as a CPU configured to execute a program and a storage such as a memory configured to store the program, and controls the operation of the pump 30. The controller 50 is configured to control the transfer of the culture medium CM by controlling the operation of the pump 30.

### (Cell Seeding Method)

The following description describes a cell seeding method in the cell culture system 100 with reference to FIGS. 2 to 6.

As shown in FIG. 2, the cell seeding method in the cell culture system 100 includes a step (S1) of filling the flow channel 13 of the cell culture device 10 with the culture medium CM, the cell culture device including the inlet 11 that is selectively fluidly connected to the inlet-side reservoir 20 and the pump 30 through the pipe 21, the outlet 12, and the flow channel 13 that is provided between the inlet 11 and the outlet 12; and a step (S2) of introducing a cell suspension CS into the flow channel 13 from the outlet 12 side of the flow channel 13 with the inlet 11 being connected to the pipe 21 after the step of filling the flow channel 13 with the culture medium CM. The cell suspension CS is a liquid in which cells to be seeded are suspended in the culture medium CM.

(Step of Filling Flow Channel with Culture Medium)

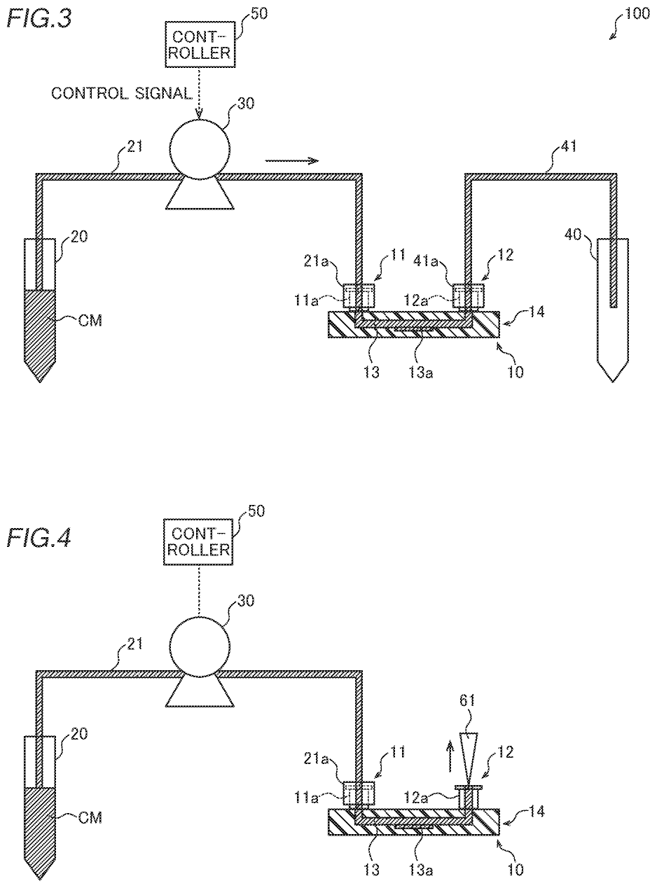

In step S1, the flow channel 13 is filled with the culture medium CM as shown in FIG. 3. Specifically, the culture medium CM is filled in the flow channel 13 by feeding the culture medium CM from the inlet 11 side to the outlet 12 side by using the pump 30 provided to the inlet 11 side of the flow channel 13. In this feeding, the culture medium CM is delivered by the pump 30 until the culture medium CM reaches the outlet-side reservoir 40 provided to the outlet 12 side of the flow channel 13. The pump 30 can be configured to stop feeding the culture medium CM immediately after the culture medium CM reaches the outlet-side reservoir 40, or to continue feeding the culture medium CM for a certain time when the culture medium CM reaches the outlet-side reservoir 40 and then stop feeding the culture medium CM. The pump 30 is configured to feed at least a predetermined amount of the culture medium CM that allows the culture medium CM to reach the outlet-side reservoir 40 based on a control signal from the controller 50.

Also, the pump 30 is configured to feed the culture medium CM at a predetermined flow rate greater than a flow rate (flow speed) in cell culture based on the control signal from the controller 50. Because cells are placed in the cell culture area 13a in cell culture, if the flow rate is too high the cells are affected, for example, are flown out of the cell culture area 13a, but because no cells are placed in the cell culture area 13a before cell seeding, even if the flow rate is increased to a certain high degree the cells are not affected, for example, are not flown out of the cell culture area 13a. In addition, it is preferable to increase the flow rate before cell seeding from the viewpoint of facilitating removal of air bubbles from the pipe 21 and the flow channel 13.

### <Step of Introducing Cell Suspension>

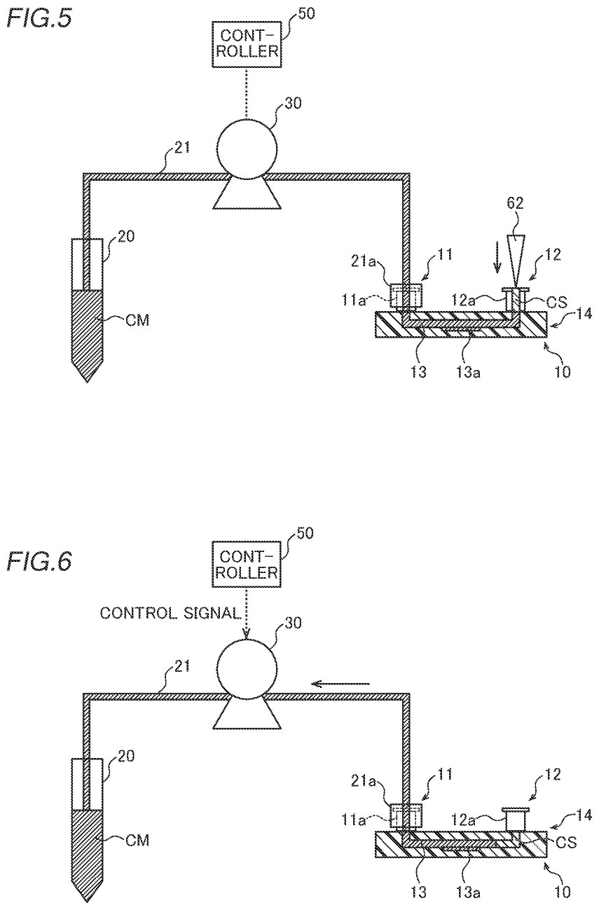

In step S2, the cell suspension CS is introduced into the flow channel 13 from the outlet 12 side of the flow channel 13, as shown in FIGS. 4 to 6. Step S2 includes a step of removing the culture medium CM from the outlet 12 of the flow channel 13 (see FIG. 4), a step of supplying the cell suspension CS from the outlet 12 of the flow channel 13 (see FIG. 5), and a step of suction the cell suspension CS (see FIG. 6).

As shown in FIG. 4, the culture medium CM is removed from the outlet 12 side of the flow channel 13.

Specifically, the outlet-side reservoir 40 and the pipe 41 connected to the outlet 12 of the flow channel 13 are first detached from the outlet 12 of the flow channel 13. That is, the connector 41a is detached from the connector 12a. The culture medium CM that is accumulated in the outlet 12 (connector 12a) of the flow channel 13 is then aspirated by an aspirator 61 such as a micropipette so that the culture medium CM is removed from the outlet 12 side of the flow channel 13. Users can detach the connector 41a from the connector 12a, and remove the culture medium CM accumulated in the outlet 12 of the flow channel 13 by using the aspirator 61.

After the culture medium CM is removed from the outlet 12 side of the flow channel 13, the cell suspension CS is supplied from the outlet 12 side of the flow channel 13 as shown in FIG. 5. Specifically, the cell suspension CS is supplied from the outlet 12 side of the flow channel 13 by discharging the cell suspension CS into space where the culture medium CM has been removed by using a discharger 62 such a micropipette. Users can supply the cell suspension CS from the outlet 12 side of the flow channel 13 by using the discharger 62.

After the cell suspension CS is supplied from the outlet 12 side of the flow channel 13, the cell suspension CS is aspirated as shown in FIG. 6. Specifically, the cell suspension CS is suctioned from the outlet 12 side to the inlet 11 side together with the culture medium CM in the flow channel 13 by the pump 30. In other words, the cell suspension CS is suctioned from the outlet 12 side to the inlet 11 side together with the culture medium CM in the flow channel 13 by rotating the pump 30 in a direction opposite to a rotation direction in which the flow channel 13 is filled with the culture medium CM. The pump 30 aspirates a predetermined amount of the culture medium CM and the cell suspension CS that can sufficiently place cells included the cell suspension CS in the cell culture area 13a based on the control signal from the controller 50. The pump 30 aspirates the predetermined amount of the culture medium CM and the cell suspension CS, which is smaller than a supply amount of the culture medium CM that has been supplied (e.g., which is a half the supply amount) based on the control signal from the controller 50.

Although a main part (part other than the connectors 11a and 12a) of the flow channel 13 provided in the device body 14 is exaggeratingly illustrated for ease of explanation, the main body of the flow channel 13 is actually much smaller than the pipe 21, the pipe 41, a flow channel part of the connector 11a, and a flow channel part of the connector 12a. For example, a volume of the main part of the flow channel 13 is approximately several micro litters, while a volume of the flow channel of the connector 12a is approximately several tens of micro litters. For this reason, when the cell suspension CS is supplied, the volume of the cell suspension CS that is supplied to the flow channel part of the connector 12a is several tens of times the volume of the main part of the flow channel 13. Also, when the cell suspension CS is suctioned, the volume of the cell suspension CS that is suctioned from the flow channel part of the connector 12a is several tens of times the volume of the main part of the flow channel 13.

Accordingly, in step S2, the cell suspension CS is introduced into the flow channel 13. Because the outlet-side reservoir 40 and the pipe 41 have been detached from the outlet 12 of the flow channel 13, the cell suspension CS is introduced from the outlet 12 of the flow channel 13 into the flow channel 13 with the outlet 12 of the flow channel 13 being opened to the outside.

As described above, in the cell seeding method in the cell culture system 100, the step (S1) of filling the flow channel 13 with the culture medium CM, and the step (S2) of introducing the cell suspension CS into the flow channel 13 are executed with the pipe 21 on the inlet 11 side with respect to the flow channel 13 being attached, in other words, without detachment of the pipe 21. In addition, the cells are seeded into the cell culture device 10 by the step (S1) of filling the flow channel 13 with the culture medium CM, and the step (S2) of introducing the cell suspension CS into the flow channel 13.

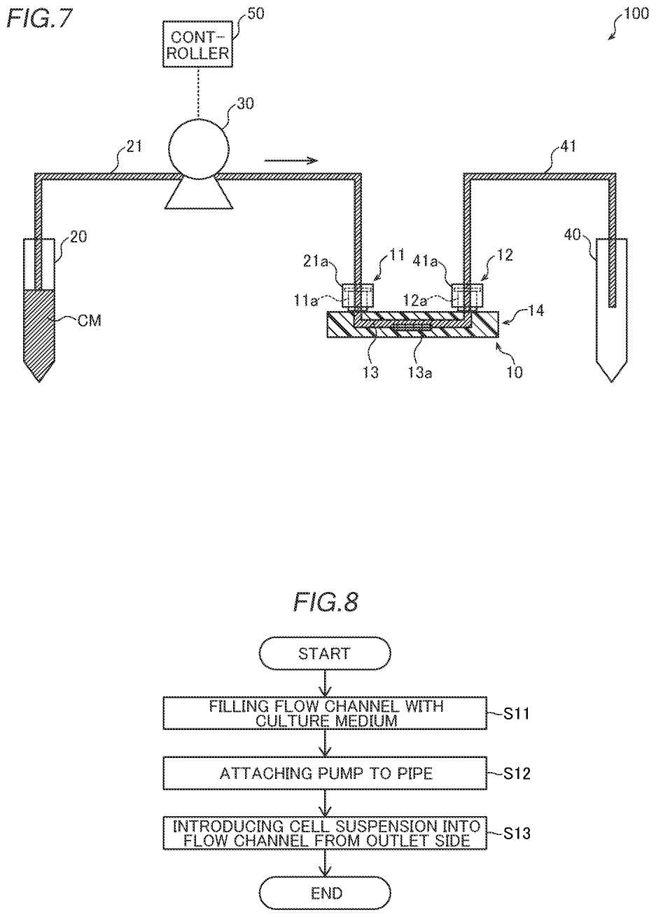

After the cells are seeded into the cell culture device 10, the outlet-side reservoir 40 and the pipe 41, which are detached, are attached to the outlet 12 of the flow channel 13, as shown in FIG. 7 so that the cells are cultivated in the cell culture area 13a. In addition, in culture of the cells, the culture medium CM is delivered from the inlet 11 side to the outlet 12 side by the pump 30. In this culture, as described later, influences of air bubbles can be suppressed, and as a result it is possible to properly cultivate the cells. Although a possibility that air bubbles enter the pipe 41 still remains in the detachment and attachment of the pipe 41, the pipe 41 is located on downstream of the cell culture area 13a so that the culture of the cells is not affected.

### (Another Cell Seeding Method)

The following description describes another cell seeding method in the cell culture system 100 with reference to FIGS. 8 to 10. In a case in which a liquid-feeding force (force for expelling air bubbles) of the pump 30 is relatively small, a cell seeding method can be performed by using a syringe 70 that has a relatively large liquid-delivering force (force for expelling air bubbles).

As shown in FIG. 8, the another cell seeding method in the cell culture system 100 includes a step (S11) of filling the flow channel 13 of the cell culture device 10 with the culture medium CM, the cell culture device including the inlet 11 that is selectively fluidly connected to the inlet-side reservoir 20 and the pump 30 through the pipe 21, the outlet 12, and the flow channel 13 that is provided between the inlet 11 and the outlet 12; and a step (S13) of introducing a cell suspension CS into the flow channel 13 from the outlet 12 side of the flow channel 13 with the inlet 11 being connected to the pipe 21 after the step of filling the flow channel 13 with the culture medium CM. The another cell seeding method in the cell culture system 100 further includes a step (S12) of attaching a pump 30 as a tube pump that is attachable to the pipe 21 without detachment of the pipe 21 after the step of filling the flow channel 13 with the culture medium CM.

(Step of Filling Flow Channel with Culture Medium)

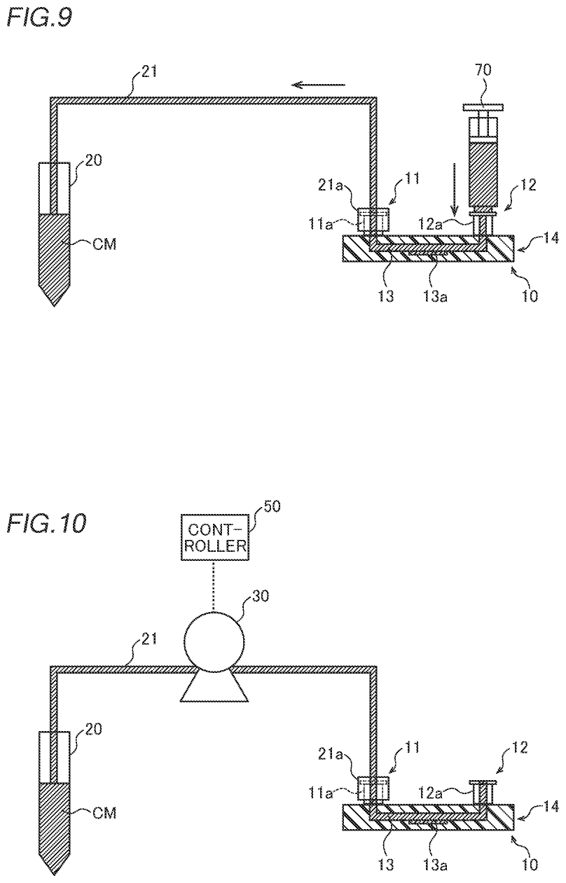

In step S11, the flow channel 13 is filled with the culture medium CM as shown in FIG. 9. Specifically, the outlet-side reservoir 40 and the pipe 41 connected to the outlet 12 of the flow channel 13 are first detached from the outlet 12 of the flow channel 13. That is, the connector 41a is detached from the connector 12a. The pump 30 is also detached from the pipe 21 connected to the inlet 11 side of the flow channel 13. A syringe 70 including the culture medium CM is attached to the outlet 12 of the flow channel 13. That is, an end of the syringe 70 is attached to the connector 12a. Subsequently, the syringe 70 provided on the outlet 12 side of the flow channel 13 fills the flow channel 13 with the culture medium CM by feeding the culture medium CM from the outlet 12 side to the inlet 11 side. In this feeding, the culture medium CM is feeded until the culture medium CM reaches at least the inlet-side reservoir 20. A user detaches the outlet-side reservoir 40 and the pipe 41 from the outlet 12 of the flow channel 13, removes the pump 30 from the pipe 21, and then attaches the syringe 70 to the outlet 12 of the flow channel 13. In addition, the user feeds the culture medium CM from the outlet 12 side to the inlet 11 side by using the syringe 70 attached. The syringe 70 is configured to be able to apply a force greater than the pump 30 to feed the culture medium.

### <Step of Attaching Pump>

In step S12, the pump 30 is attached to the pipe 21 as shown in FIG. 10. Specifically, the pump 30 is attached to the pipe 21 so as to nip outer circumferential part of the pipe 21 without detachment of the pipe 21, in other words, with the pipe 21 being connected. The user attached the pump 30 to the pipe 21.

### <Step of Introducing Cell Suspension>

In step S13, the cell suspension CS is introduced into the flow channel 13 from the outlet 12 side of the flow channel 13. Because step S13 is similar to step S2, and includes a step of removing the culture medium CM from the outlet 12 of the flow channel 13 (see FIG. 4), a step of supplying the cell suspension CS from the outlet 12 of the flow channel 13 (see FIG. 5), and a step of suction the cell suspension CS (see FIG. 6), its description is omitted.

As described above, in the another cell seeding method in the cell culture system 100, the step (S11) of filling the flow channel 13 with the culture medium CM, the step (S12) of attaching the pump 30 to the pipe 21, and the step (S13) of introducing the cell suspension CS into the flow channel 13 are executed with the pipe 21 on the inlet 11 side with respect to the flow channel 13 being attached, in other words, without detachment of the pipe 21. In addition, the cells are seeded into the cell culture device 10 by the step (S11) of filling the flow channel 13 with the culture medium CM, the step (S12) of attaching the pump 30 to the pipe 21, and the step (S13) of introducing the cell suspension CS into the flow channel 13.

### Advantages of First Embodiment

In the first embodiment, the following advantages are obtained.

In the first embodiment, as described above, the step S1 or S11 of filling the flow channel 13 with the culture medium CM is provided. Accordingly, air bubbles can be removed by feeding the culture medium CM when filling the flow channel 13 with the culture medium CM. In addition, the step S2 or S13 of introducing the cell suspension CS into the flow channel 13 from the outlet 12 side of the flow channel 13 with the inlet 11 being connected to the pipe 21 after the step S1 or S11 of filling the flow channel 13 with the culture medium CM is provided. As a result, the cell suspension CS (cells) can be introduced without detachment/attachment of the pipe 21 to/from the inlet 11 side of the flow channel 13 dissimilar to in a case in which the cell suspension CS is introduced from the inlet 11 side of the flow channel 13. Consequently, it is possible to remove air bubbles caused by detachment/attachment of the pipe 21 to/from the inlet 11 side of the flow channel 13. Therefore, it is possible to suppress influences of the air bubbles and properly perform cell culture.

In the first embodiment, as described above, the step S1 or S11 of filling the flow channel 13 with the culture medium CM, and the step S2 or S13 of introducing the cell suspension CS into the flow channel 13 are executed with the pipe 21 on the inlet 11 side with respect to the flow channel 13 being attached, in other words, without detachment of the pipe 21. Accordingly, because cells contained in the cell suspension CS can be placed in the cell culture area 13a without detachment of the pipe 21 on the inlet 11 side with respect to the flow channel 13, it is possible to reliably remove air bubbles caused by detachment/attachment of the pipe 21 to/from the inlet 11 side of the flow channel 13.

In the first embodiment, as described above, the step S1 of filling the flow channel 13 with the culture medium CM includes a step of feeding the culture medium CM from the inlet 11 side to the outlet 12 by using the pump 30 that is provided to the inlet 11 side of the flow channel 13 to fill the flow channel 13 with the culture medium CM; and the step S2 of introducing the cell suspension CS into the flow channel 13 includes a step of aspirating the culture medium CM and the cell suspension CS from the outlet 12 side to the inlet 11 side by using the pump 30 to introduce the cell suspension CS into the flow channel 13. Accordingly, because the same pump 30 can be effectively used to execute the step S1 of filling the flow channel 13 with the culture medium CM and the step S2 of introducing the cell suspension CS into the flow channel 13, an advantage of properly cultivating cells can be obtained while suppressing influences of air bubbles by a simple configuration.

In the first embodiment, as described above, the step S1 of filling the flow channel 13 with the culture medium CM includes a step of delivering the culture medium CM by using the pump 30 to bring the culture medium CM to reach the outlet-side reservoir 40 provided to the outlet 12 side of the flow channel 13 so as to fill the flow channel 13 with the culture medium CM. Accordingly, because the culture medium CM can be sufficiently feeded into the flow channel 13, air bubbles that exist upstream of the cell culture area 13a can be reliably expelled by feeding the culture medium CM.

In the first embodiment, as described above, the step S2 or S13 of introducing the cell suspension CS into the flow channel 13 includes a step of introducing the cell suspension CS from the outlet 12 side of the flow channel 13 into the flow channel 13 with the outlet 12 side of the flow channel 13 being opened. Accordingly, introduction of air bubbles from the outlet 12 side of the flow channel 13 to the outlet 12 side of the flow channel 13 can be suppressed by effectively using an effect of opening of the outlet 12 side of the flow channel 13 that completely or almost completely prevents appearance of air bubbles in the introduction of the cell suspension CS into the flow channel 13 from the outlet 12 side of the flow channel 13. Consequently, it is possible to further suppress influences of air bubbles.

In the first embodiment, as described above, the step S11 of filling the flow channel 13 with the culture medium CM includes a step of feeding the culture medium CM from the outlet 12 side to the inlet 11 side by using the syringe 70 provided to the outlet 12 side of the flow channel 13 to fill the flow channel 13 with the culture medium CM. Also, according to this configuration, air bubbles can be removed by feeding the culture medium CM when filling the flow channel 13 with the culture medium CM. In addition, even if a liquid-feeding force of the pump 30 on the inlet 11 side is relatively small so that air bubbles cannot be sufficiently expelled by feeding of the culture medium CM, the air bubbles can be sufficiently expelled by the syringe 70 on the outlet 12 side whose liquid-feeding force is greater force than the pump 30.

In the first embodiment, as described above, the step S12 of attaching the pump 30 as a tube pump that is attachable to the pipe 21 without detachment of the pipe 21 after the step S11 of filling the flow channel 13 with the culture medium CM is further provided. Accordingly, because the pump 30 (tube pump) can be attached without detaching/attaching the pipe 21 on the inlet 11 side of the flow channel 13 even in a case in which the step of filling the flow channel 13 with the culture medium CM is executed by using the syringe 70 on the outlet 12 side, the culture medium CM can be delivered by the attached pump 30 while removing air bubbles caused by detachment/attachment of the pipe 21 on the inlet 11 side of the flow channel 13.

Also, in the first embodiment, as mentioned above, the cell culture element is arranged in a position in the flow channel 13 that is not directly exposed to the outside. In a case in which the cell culture element is arranged in a position that is not directly exposed to the outside, air bubbles cannot be easily removed so that culture of cells is likely to be affected by the air bubbles. For this reason, the advantage of properly cultivating cells while suppressing influences of air bubbles by a simple configuration is very effective in this case.

### Second Embodiment

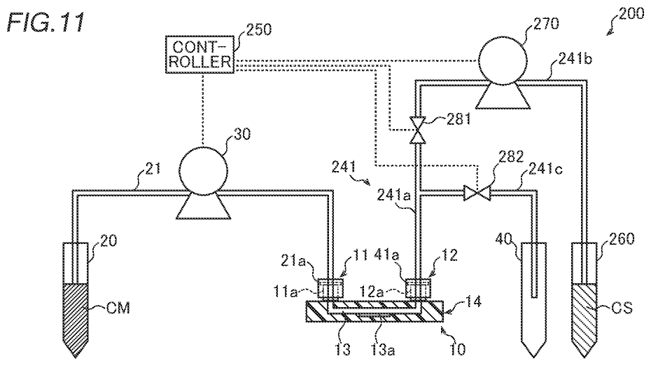

The following description describes a cell culture system according to a second embodiment with reference to FIG. 11. In the second embodiment, the controller is configured to execute the step of filling a flow channel with a culture medium, and the step of introducing a cell suspension into the flow channel. The same components as those of the first embodiment are denoted by the same reference numerals, and their description is omitted.

### (Overall Configuration of Cell Culture System)

As shown in FIG. 11, the cell culture system 200 includes a cell culture device 10, an inlet-side reservoir 20, a pump 30, an outlet-side reservoir 40, and a controller 250, a cell suspension reservoir 260, and a pump 270. The controller 250 is an example of a “controller” in the claims. The cell suspension reservoir 260 and the pump 270 are examples of a “cell supplier” in the claims.

The cell suspension reservoir 260 is configured for storage of a cell suspension CS supplied to the cell culture device 10. The cell suspension reservoir 260 is a container such as a conical tube. The cell suspension reservoir 260 is selectively fluidly connected to the outlet 12 of the cell culture device 10 through the pipe 241. Specifically, the pipe 241 includes a merging flow channel part 241a, and branching flow channel parts 241b and 241c that branch from the merging flow channel part 241a. The cell suspension reservoir 260 is selectively fluidly connected to the outlet 12 of the cell culture device 10 through the branching flow channel part 241b of the pipe 241. Also, the outlet-side reservoir 40 is selectively fluidly connected to the outlet 12 of the cell culture device 10 through the branching flow channel part 241c of the pipe 241. The branching flow channel parts 241b and 241c include valves 281 and 282, respectively. The valves 281 and 282 are configured to open/close the branching flow channel parts 241b and 241c, respectively. The valves 281 and 282 are solenoid valves configured to be controlled by the controller 250 to open/close the branching flow channel parts.

The pipe 241 is a flexible tube formed from a material such as resin or rubber. An end of the branching flow channel part 241b of the pipe 241 is arranged in the cell suspension reservoir 260, and is immersed in the cell suspension CS that is stored in the cell suspension reservoir 260. An end of the branching flow channel part 241c of the pipe 241 is arranged in the outlet-side reservoir 40. An end of the merging flow channel part 241a of the pipe 241 is connected to the outlet 12 of the cell culture device 10. Specifically, a connector 41a is provided on the end of merging flow channel part 241a of the pipe 241, and the connector 41a is connected to a connector 12a that is provided on the outlet 12 of the cell culture device 10.

The pump 270 delivers the cell suspension CS. The pump 270 is provided to the outlet 12 side of the flow channel 13 of the cell culture device 10. Specifically, the pump 270 is attached to the branching flow channel part 241b of the pipe 241. The pump 270 is a tube pump (peristaltic pump) configured to deliver the cell suspension CS by pressing the branching flow channel part 241b of the pipe 241. The cell suspension reservoir 260 and the pump 270 serve as a cell supplier for supplying the cell suspension CS to the cell culture device 10.

The controller 250 includes a processor such as a CPU configured to execute a program and a storage such as a memory configured to store the program, and controls operations of the pumps 30 and 270, and the valves 281 and 282. The controller 250 is configured to control the delivery of the culture medium CM by controlling the operation of the pump 30. The controller 250 is configured to control the delivery of the cell suspension CS by controlling the operation of the pump 270.

In the second embodiment, the controller 250 is configured to execute a step (process) of filling the flow channel 13 with the culture medium CM, and a step (process) of introducing the cell suspension CS into the flow channel 13 from the outlet 12 side of the flow channel 13 with the inlet 11 being connected to the pipe 21 after the step of filling the flow channel 13 with the culture medium CM when controlling the operations. The steps are similar to the steps in the aforementioned first embodiment except that they are automatically executed by the controller 250. For this reason, differences are only explained.

(Step of Filling Flow Channel with Culture Medium)

In the step of filling the flow channel 13 with the culture medium CM, the culture medium CM is filled in the flow channel 13 by feeding the culture medium CM from the inlet 11 side to the outlet 12 side by using the pump 30 based on a control signal from the controller 250. In this step, the valve 281 is closed while the valve 282 is opened based on the control signal from the controller 250. As a result, the culture medium CM flows through the branching flow channel part 241c of the pipe 241 to the outlet-side reservoir 40.

### <Step of Introducing Cell Suspension>

In the step of introducing the cell suspension CS, the cell suspension CS is introduced from the outlet 12 side of the flow channel 13 into the flow channel 13 by delivering the cell suspension CS from the outlet 12 side to the inlet 11 side by using the pump 270 based on the control signal from the controller 250. In this step, the valve 281 is opened while the valve 282 is closed based on the control signal from the controller 250. As a result, the cell suspension CS flows through the branching flow channel part 241b of the pipe 241 from the cell suspension reservoir 260 to the cell culture device 10.

### Advantages of Second Embodiment

In the second embodiment, the following advantages are obtained.

In the second embodiment, as described above, the controller 250 is configured to execute a step of filling the flow channel 13 with the culture medium CM, and a step of introducing the cell suspension CS into the flow channel 13 from the outlet 12 side of the flow channel 13 with the inlet 11 being connected to the pipe 21 after the step of filling the flow channel 13 with the culture medium CM. Accordingly, similar to the above first embodiment, it is possible to suppress influences of the air bubbles and properly perform cell culture.

The other advantages of the second embodiment are similar to the first embodiment.

### Modified Embodiments

Note that the embodiment disclosed this time must be considered as illustrative in all points and not restrictive. The scope of the present invention is not shown by the above description of the embodiments but by the scope of claims for patent, and all modifications (modified examples) within the meaning and scope equivalent to the scope of claims for patent are further included.

While the example in which the cell seeding method includes both automated and manual process, which are performed by the controller and by the operator respectively, has been shown in the aforementioned first embodiment, and the example in which the cell seeding method includes only automated processes has been shown in the aforementioned second embodiment, the present invention is not limited to these. Alternatively, in the present invention, the cell seeding method can realized by fully automated, semi-automated, or fully manual processing. In a case of the semi-automatic processing, each process can be automatically or manually executed, in other words, the automated or manual process is not limited to a particular process.

While the example in which the culture medium is transfered by the pump until the culture medium reaches the outlet-side reservoir has been shown in the aforementioned first embodiment, the present invention is not limited to this. In the present invention, the culture medium can be delivered by the pump with the pipe on the outlet side of the flow channel and the outlet-side reservoir being detached from the outlet side of the flow channel. In this case, the culture medium can be pumped by the pump while the culture medium is aspirated from the outlet of the flow channel by an aspirator such as a pipette.

While the example in which the flow channel is filled with the culture medium by feeding of the culture medium by using the pump, and the example in which the flow channel is filled with the culture medium by feeding of the culture medium by using the syringe have been shown in the aforementioned first embodiment, the present invention is not limited to these.

While the example in which the pump is a tube pump has been shown in the aforementioned first and second embodiments, the present invention is not limited to this. In the present invention, the pump can be a syringe pump.

While the example in which the cell suspension is introduced into the flow channel from the outlet side of the flow channel by delivering the cell suspension by the pump provided to the outlet side of the flow channel has been shown in the aforementioned second embodiment, the present invention is not limited to this. In the second embodiment above, the cell suspension can be introduced into the flow channel from the outlet side of the flow channel by aspirating the cell suspension by a pump provided to the inlet side of the flow channel (i.e., pump 30).

While the example in which the cell culture device has two ports, which are the inlet (first port) and the outlet (second port) has been shown in the aforementioned first and second embodiments, the present invention is not limited to this. In the present invention, the cell culture device can have a third or subsequent ports (three or more ports).

### [Modes]

The aforementioned exemplary embodiment will be understood as concrete examples of the following modes by those skilled in the art.

### (Mode Item 1)

A cell seeding method according to mode item 1 includes a step of filling a flow channel of a cell culture device with a culture medium, the cell culture device including a first port that is selectively fluidly connected to a culture medium supplier through a pipe, a second port, and the flow channel that is provided between the first and second ports; and a step of introducing a cell suspension into the flow channel from the second port side of the flow channel with the first port being connected to the pipe after the step of filling the flow channel with the culture medium.

### (Mode Item 2)

In the cell seeding method according to mode item 1, the step of filling the flow channel with the culture medium includes a step of feeding the culture medium from the first port side to the second port side by using a pump that is provided to the first port side of the flow channel to fill the flow channel with the culture medium; and the step of introducing the cell suspension into the flow channel includes a step of drawing the culture medium and the cell suspension from the second port side to the first port side by using the pump to introduce the cell suspension into the flow channel.

### (Mode Item 3)

In the cell seeding method according to mode item 2, the step of filling the flow channel with the culture medium includes a step of feeding the culture medium by using the pump to bring the culture medium to reach a second-port side reservoir that is provided to the second port side of the flow channel so as to fill the flow channel with the culture medium.

### (Mode Item 4)

In the cell seeding method according to any of mode items 1 to 3, the step of introducing the cell suspension into the flow channel includes a step of introducing the cell suspension from the second port side of the flow channel into the flow channel with the second port side of the flow channel being opened.

### (Mode Item 5)

In the cell seeding method according to any of mode items 1 to 4, the step of filling the flow channel with the culture medium includes a step of feeding the culture medium from the second port side to the first port side by using a syringe that is provided to the second port side of the flow channel so as to fill the flow channel with the culture medium.

### (Mode Item 6)

In the cell seeding method according to mode item 5, a step of attaching a tube pump that is attachable to the pipe without detachment of the pipe after the step of filling the flow channel with the culture medium is further provided.

### (Mode Item 7)

In the cell seeding method according to any of mode items 1 to 6, a cell culture element is arranged in a position in the flow channel that is not directly exposed to the outside.

### (Mode Item 8)

A cell culture system according mode item 8 includes a cell culture device including a first port, a second port, and a flow channel that is provided between the first and second ports; a pipe that is connected to the first port; a culture medium supplier that is selectively fluidly connected to the first port via the pipe; a cell supplier that is selectively fluidly connected to the second port, and a controller, wherein the controller is configured to execute a step of filling the flow channel with a culture medium, and a step of introducing a cell suspension into the flow channel from the second port side of the flow channel with the first port being connected to the pipe after the step of filling the flow channel with the culture medium.

What is claimed is:

1. A cell seeding method comprising:
a step of filling a flow channel of a cell culture device with a culture medium, the cell culture device including a first port that is selectively fluidly connected to a culture medium supplier through a pipe, a second port, and the flow channel that is provided between the first and second ports; and
a step of introducing a cell suspension into the flow channel from the second port side of the flow channel with the first port being connected to the pipe after the step of filling the flow channel with the culture medium.

2. The cell seeding method according to claim 1, wherein
the step of filling the flow channel with the culture medium includes a step of feeding the culture medium from the first port side to the second port side by using a pump that is provided to the first port side of the flow channel to fill the flow channel with the culture medium; and
the step of introducing the cell suspension into the flow channel includes a step of drawing the culture medium and the cell suspension from the second port side to the first port side by using the pump to introduce the cell suspension into the flow channel.

3. The cell seeding method according to claim 2, wherein the step of filling the flow channel with the culture medium includes a step of feeding the culture medium by using the pump to bring the culture medium to reach a second-port side reservoir that is provided to the second port side of the flow channel so as to fill the flow channel with the culture medium.

4. The cell seeding method according to claim 1, wherein the step of introducing the cell suspension into the flow channel includes a step of introducing the cell suspension from the second port side of the flow channel into the flow channel with the second port side of the flow channel being opened.

5. The cell seeding method according to claim 1, wherein the step of filling the flow channel with the culture medium includes a step of delivering the culture medium from the second port side to the first port side by using a syringe that is provided to the second port side of the flow channel so as to fill the flow channel with the culture medium.

6. The cell seeding method according to claim 5 further comprising a step of attaching a tube pump that is attachable to the pipe without detachment of the pipe after the step of filling the flow channel with the culture medium.

7. The cell seeding method according to claim 1, wherein a cell culture element is arranged in a position in the flow channel that is not directly exposed to the outside.

8. A cell culture system comprising:
a cell culture device including a first port, a second port, and a flow channel that is provided between the first and second ports;
a pipe that is connected to the first port;
a culture medium supplier that is selectively fluidly connected to the first port via the pipe;
a cell supplier that is selectively fluidly connected to the second port, and
a controller, wherein
the controller is configured execute
a step of filling the flow channel with a culture medium, and
a step of introducing a cell suspension into the flow channel from the second port side of the flow channel with the first port being connected to the pipe after the step of filling the flow channel with the culture medium.
